SEQUENCE LISTING (23 sequences)

SEQ ID NO: 1 - protein, 2556 aa, Homo sapiens
  misc_feature (891)..(891): Xaa can be any naturally occurring amino acid
MPPLLAPLLCLALLPALAARGPRCSQPGETCLNGGKCEAANGTEACVCGGAFVGPRCQDPNPCLSTPCKNAGTCHVVDRRGVADYACSCALGFSGPLCLTPLDNACLTNPCRNGGTCDLLTLTEYKCRCPPGWSGKSCQQADPCASNPCANGGQCLPFEASYICHCPPSFHGPTCRQDVNECGQKPRLCRHGGTCHNEVGSYRCVCRATHTGPNCERPYVPCSPSPCQNGGTCRPTGDVTHECACLPGFTGQNCEENIDDCPGNNCKNGGACVDGVNTYNCPCPPEWTGQYCTEDVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCMCMPGYEGVHCEVNTDECASSPCLHNGRCLDKINEFQCECPTGFTGHLCQYDVDECASTPCKNGAKCLDGPNTYTCVCTEGYTGTHCEVDIDECDPDPCHYGSCKDGVATFTCLCRPGYTGHHCETNINECSSQPCRLRGTCQDPDNAYLCFCLKGTTGPNCEINLDDCASSPCDSGTCLDKIDGYECACEPGYTGSMCNSNIDECAGNPCHNGGTCEDGINGFTCRCPEGYHDPTCLSEVNECNSNPCVHGACRDSLNGYKCDCDPGWSGTNCDINNNECESNPCVNGGTCKDMTSGIVCTCREGFSGPNCQTNINECASNPCLNKGTCIDDVAGYKCNCLLPYTGATCEVVLAPCAPSPCRNGGECRQSEDYESFSCVCPTAGAKGQTCEVDINECVLSPCRHGASCQNTHGXYRCHCQAGYSGRNCETDIDDCRPNPCHNGGSCTDGINTAFCDCLPGFRGTFCEEDINECASDPCRNGANCTDCVDSYTCTCPAGFSGIHCENNTPDCTESSCFNGGTCVDGINSFTCLCPPGFTGSYCQHVVNECDSRPCLLGGTCQDGRGLHRCTCPQGYTGPNCQNLVHWCDSSPCKNGGKCWQTHTQYRCECPSGWTGLYCDVPSVSCEVAAQRQGVDVARLCQHGGLCVDAGNTHHCRCQAGYTGSYCEDLVDECSPSPCQNGATCTDYLGGYSCKCVAGYHGVNCSEEIDECLSHPCQNGGTCLDLPNTYKCSCPRGTQGVHCEINVDDCNPPVDPVSRSPKCFNNGTCVDQVGGYSCTCPPGFVGERCEGDVNECLSNPCDARGTQNCVQRVNDFHCECRAGHTGRRCESVINGCKGKPCKNGGTCAVASNTARGFICKCPAGFEGATCENDARTCGSLRCLNGGTCISGPRSPTCLCLGPFTGPECQFPASSPCLGGNPCYNQGTCEPTSESPFYRCLCPAKFNGLLCHILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADVQPLGPSSLAVHTILPQESPALPTSLPSSLVPPVTAAQFLTPPSQHSYSSPVDNTPSHQLQVPEHPFLTPSPESPDQWSSSSPHSNVSDWSEGVSSPPTSMQSQIARIPEAFK

SEQ ID NO: 2 - protein, 2471 aa, Homo sapiens
MPALRPALLWALLALWLCCAAPAHALQCRDGYEPCVNEGMCVTYHNGTGYCKCPEGFLGEYCQHRDPCEKNRCQNGGTCVAQAMLGKATCRCASGFTGEDCQYSTSHPCFVSRPCLNGGTCHMLSRDTYECTCQVGFTGKECQWTDACLSHPCANGSTCTTVANQFSCKCLTGFTGQKCETDVNECDIPGHCQHGGTCLNLPGSYQCQCPQGFTGQYCDSLYVPCAPSPCVNGGTCRQTGDFTFECNCLPGFEGSTCERNIDDCPNHRCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCELEINECQSNPCVNNGQCVDKVNRFQCLCPPGFTGPVCQIDIDDCSSTPCLNGAKCIDHPNGYECQCATGFTGVLCEENIDNCDPDPCHHGQCQDGIDSYTCICNPGYMGAICSDQIDECYSSPCLNDGRCIDLVNGYQCNCQPGTSGVNCEINFDDCASNPCIHGICMDGINRYSCVCSPGFTGQRCNIDIDECASNPCRKGATCINGVNGFRCICPEGPHHPSCYSQVNECLSNPCIHGNCTGGLSGYKCLCDAGWVGINCEVDKNECLSNPCQNGGTCDNLVNGYRCTCKKGFKGYNCQVNIDECASNPCLNQGTCFDDISGYTCHCVLPYTGKNCQTVLAPCSPNPCENAAVCKESPNFESYTCLCAPGWQGQRCTIDIDECISKPCMNHGLCHNTQGSYMCECPPGFSGMDCEEDIDDCLANPCQNGGSCMDGVNTFSCLCLPGFTGDKCQTDMNECLSEPCKNGGTCSDYVNSYTCKCQAGFDGVHCENNINECTESSCFNGGTCVDGINSFSCLCPVGFTGSFCLHEINECSSHPCLNEGTCVDGLGTYRCSCPLGYTGKNCQTLVNLCSRSPCKNKGTCVQKKAESQCLCPSGWAGAYCDVPNVSCDIAASRRGVLVEHLCQHSGVCINAGNTHYCQCPLGYTGSYCEEQLDECASNPCQHGATCSDFIGGYRCECVPGYQGVNCEYEVDECQNQPCQNGGTCIDLVNHFKCSCPPGTRGLLCEENIDDCARGPHCLNGGQCMDRIGGYSCRCLPGFAGERCEGDINECLSNPCSSEGSLDCIQLTNDYLCVCRSAFTGRHCETFVDVCPQMPCLNGGTCAVASNMPDGFICRCPPGFSGARCQSSCGQVKCRKGEQCVHTASGPRCFCPSPRDCESGCASSPCQHGGSCHPQRQPPYYSCQCAPPFSGSRCELYTAPPSTPPATCLSQYCADKARDGVCDEACNSHACQWDGGDCSLTMENPWANCSSPLPCWDYINNQCDELCNTVECLFDNFECQGNSKTCKYDKYCADHFKDNHCNQGCNSEECGWDGLDCAADQPENLAEGTLVIVVLMPPEQLLQDARSFLRALGTLLHTNLRIKRDSQGELMVYPYYGEKSAAMKKQRMTRRSLPGEQEQEVAGSKVFLEIDNRQCVQDSDHCFKNTDAAAALLASHAIQGTLSYPLVSVVSESLTPERTQLLYLLAVAVVIILFIILLGVIMAKRKRKHGSLWLPEGFTLRRDASNHKRREPVGQDAVGLKNLSVQVSEANLIGTGTSEHWVDDEGPQPKKVKAEDEALLSEEDDPIDRRPWTQQHLEAADIRRTPSLALTPPQAEQEVDVLDVNVRGPDGCTPLMLASLRGGSSDLSDEDEDAEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDRMHHDIVRLLDEYNVTPSPPGTVLTSALSPVICGPNRSFLSLKHTPMGKKSRRPSAKSTMPTSLPNLAKEAKDAKGSRRKKSLSEKVQLSESSVTLSPVDSLESPHTYVSDTTSSPMITSPGILQASPNPMLATAAPPAPVHAQHALSFSNLHEMQPLAHGASTVLPSVSQLLSHHHIVSPGSGSAGSLSRLHPVPVPADWMNRMEVNETQYNEMFGMVLAPAEGTHPGIAPQSRPPEGKHITTPREPLPPIVTFQLIPKGSIAQPAGAPQPQSTCPPAVAGPLPTMYQIPEMARLPSVAFPTAMMPQQDGQVAQTILPAYHPFPASVGKYPTPPSQHSYASSNAAERTPSHSGHLQGEHPYLTPSPESPDQWSSSSPHSASDWSDVTTSPTPGGAGGGQRGPGTHMSEPPHNNMQVYA

SEQ ID NO: 3 - protein, 2281 aa, Homo sapiens
PPCLDGSPCANGGRCTQLPSREAACLCPPGWVGERCQLEDPCHSGPCAGRGVCQSSVVAGTARFSCRCPRGFRGPDCSLPDPCLSSPCAHGARCSVGPDGRFLCSCPPGYQGRSCRSDVDECRVGEPCRHGGTCLNTPGSFRCQCPAGYTGPLCENPAVPCAPSPCRNGGTCRQSGDLTYDCACLPGFEGQNCEVNVDDCPGHRCLNGGTCVDGVNTYNCQCPPEWTGQFCTEDVDECQLQPNACHNGGTCFNTLGGHSCVCVNGWTGESCSQNIDDCATAVCFHGATCHDRVASFYCACPMGKTGLLCHLDDACVSNPCHEDAICDTNPVNGRAICTCPPGFTGGACDQDVDECSIGANPCEHLGRCVNTQGSFLCQCGRGYTGPRCETDVNECLSGPCRNQATCLDRIGQFTCICMAGFTGTYCEVDIDECQSSPCVNGGVCKDRVNGFSCTCPSGFSGSTCQLDVDECASTPCRNGAKCVDQPDGYECRCAEGFEGTLCDRNVDDCSPDPCHHGRCVDGIASFSCACAPGYTGTRCESQVDECRSQPCRHGGKCLDLVDKYLCRCPSGTTGVNCEVNIDDCASNPCTFGVCRDGINRYDCVCQPGFTGPLCNVEINECASSPCGEGGSCVDGENGFRCLCPPGSLPPLCLPPSHPCAHEPCSHGICYDAPGGFRCVCEPGWSGPRCSQSLARDACESQPCRAGGTCSSDGMGFHCTCPPGVQGRQCELLSPCTPNPCEHGGRCESAPGQLPVCSCPQGWQGPRCQQDVDECAGPAPCGPHGICTNLAGSFSCTCHGGYTGPSCDQDINDCDPNPCLNGGSCQDGVGSFSCSCLPGFAGPRCARDVDECLSNPCGPGTCTDHVASFTCTCPPGYGGFHCEQDLPDCSPSSCFNGGTCVDGVNSFSCLCRPGYTGAHCQHEADPCLSRPCLHGGVCSAAHPGFRCTCLESFTGPQCQTLVDWCSRQPCQNGGRCVQTGAYCLCPPGWSGRLCDIRSLPCREAAAQIGVRLEQLCQAGGQCVDEDSSHYCVCPEGRTGSHCEQEVDPCLAQPCQHGGTCRGYMGGYMCECLPGYNGDNCEDDVDECASQPCQHGGSCIDLVARYLCSCPPGTLGVLCEINEDDCGPGPPLDSGPRCLHNGTCVDLVGGFRCTCPPGYTGLRCEADINECRSGACHAAHTRDCLQDPGGGFRCLCHAGFSGPRCQTVLSPCESQPCQHGGQCRPSPGPGGGLTFTCHCAQPFWGPRCERVARSCRELQCPVGVPCQQTPRGPRCACPPGLSGPSCRSFPGSPPGASNASCAAAPCLHGGSCRPAPLAPFFRCACAQGWTGPRCEAPAAAPEVSEEPRCPRAACQAKRGDQRCDRECNSPGCGWDGGDCSLSVGDPWRQCEALQCWRLFNNSRCDPACSSPACLYDNFDCHAGGRERTCNPVYEKYCADHFADGRCDQGCNTEECGWDGLDCASEVPALLARGVLVLTVLLPPEELLRSSADFLQRLSAILRTSLRFRLDAHGQAMVFPYHRPSPGSEPRARRELAPEVIGSVVMLEIDNRLCLQSPENDHCFPDAQSAADYLGALSAVERLDFPYPLRDVRGEPLEPPEPSVPLLPLLVAGAVLLLVILVLGVMVARRKREHSTLWFPEGFSLHKDVASGHKGRREPVGQDALGMKNMAKGESLMGEVATDWMDTECPEAKRLKVEEPGMGAEEAVDCRQWTQHHLVAADIRVAPAMALTPPQGDADADGMDVNVRGPDGFTPLMLASFCGGALEPMPTEEDEADDTSASIISDLICQGAQLGARTDRTGETALHLAARYARADAAKRLLDAGADTNAQDHSGRTPLHTAVTADAQGVFQILIRNRSTDLDARMADGSTALILAARLAVEGMVEELIASHADVNAVDELGKSALHWAAAVNNVEATLALLKNGANKDMQDSKEETPLFLAAREGSYEAAKLLLDHFANREITDHLDRLPRDVAQERLHQDIVRLLDQPSGPRSPPGPHGLGPLLCPPGAFLPGLKAAQSGSKKSRRPPGKAGLGPQGPRGRGKKLTLACPGPLADSSVTLSPVDSLDSPRPFGGPPASPGGFPLEGPYAAATATAVSLAQLGGPGRAGLGRQPPGGCVLSLGLLNPVAVPLDWARLPPPAPPGPSFLLPLAPGPQLLNPGTPVSPQERPPPYLAVPGHGEEYPAAGAHSSPPKARFLRVPSEHPYLTPSPESPEHWASPSPPSLSDWSESTPSPATATGAMATTTGALPAQPLPLSVPSSLAQAQTQLGPQPEVTPKRQVLA

SEQ ID NO: 4 - protein, 2003 aa, Homo sapiens
MQPPSLLLLLLLLLLLCVSVVRPRGLLCGSFPEPCANGGTCLSLSLGQGTCQCAPGFLGETCQFPDPCQNAQLCQNGGSCQALLPAPLGLPSSPSPLTPSFLCTCLPGFTGERCQAKLEDPCPPSFCSKRGRCHIQASGRPQCSCMPGWTGEQCQLRDFCSANPCVNGGVCLATYPQIQCHCPPGFEGHACERDVNECFQDPGPCPKGTSCHNTLGSFQCLCPVGQEGPRCELRAGPCPPRGCSNGGTCQLMPEKDSTFHLCLCPPGFIGPDCEVNPDNCVSHQCQNGGTCQDGLDTYTCLCPETWTGWDCSEDVDECETQGPPHCRNGGTCQNSAGSFHCVCVSGWGGTSCEENLDDCIAATCAPGSTCIDRVGSFSCLCPPGRTGLLCHLEDMCLSQPCHGDAQCSTNPLTGSTLCLCQPGYSGPTCHQDLDECLMAQQGPSPCEHGGSCLNTPGSFNCLCPPGYTGSRCEADHNECLSQPCHPGSTCLDLLATFHCLCPPGLEGQLCEVETNECASAPCLNHADCHDLLNGFQCICLPGFSGTRCEEDIDECRSSPCANGGQCQDQPGAFHCKCLPGFEGPRCQTEVDECLSDPCPVGASCLDLPGAFFCLCPSGFTGQLCEVPLCAPNLCQPKQICKDQKDKANCLCPDGSPGCAPPEDNCTCHHGHCQRSSCVCDVGWTGPECEAELGGCISAPCAHGGTCYPQPSGYNCTCPTGYTGPTCSEEMTACHSGPCLNGGSCNPSPGGYYCTCPPSHTGPQCQTSTDYCVSAPCFNGGTCVNRPGTFSCLCAMGFQGPRCEGKLRPSCADSPCRNRATCQDSPQGPRCLCPTGYTGGSCQTLMDLCAQKPCPRNSHCLQTGPSFHCLCLQGWTGPLCNLPLSSCQKAALSQGIDVSSLCHNGGLCVDSGPSYFCHCPPGFQGSLCQDHVNPCESRPCQNGATCMAQPSGYLCQCAPGYDGQNCSKELDACQSQPCHNHGTCTPKPGGFHCACPPGFVGLRCEGDVDECLDQPCHPTGTAACHSLANAFYCQCLPGHTGQWCEVEIDPCHSQPCFHGGTCEATAGSPLGFICHCPKGFEGPTCSHRAPSCGFHHCHHGGLCLPSPKPGFPPRCACLSGYGGPDCLTPPAPKGCGPPSPCLYNGSCSETTGLGGPGFRCSCPHSSPGPRCQKPGAKGCEGRSGDGACDAGCSGPGGNWDGGDCSLGVPDPWKGCPSHSRCWLLFRDGQCHPQCDSEECLFDGYDCETPPACTPAYDQYCHDHFHNGHCEKGCNTAECGWDGGDCRPEDGDPEWGPSLALLVVLSPPALDQQLFALARVLSLTLRVGLWVRKDRDGRDMVYPYPGARAEEKLGGTRDPTYQERAAPQTQPLGKETDSLSAGFVVVMGVDLSRCGPDHPASRCPWDPGLLLRFLAAMAAVGALEPLLPGPLLAVHPHAGTAPPANQLPWPVLCSPVAGVILLALGALLVLQLIRRRRREHGALWLPPGFTRRPRTQSAPHRRRPPLGEDSIGLKALKPKAEVDEDGVVMCSGPEEGEEVGQAEETGPPSTCQLWSLSGGCGALPQAAMLTPPQESEMEAPDLDTRGPDGVTPLMSAVCCGEVQSGTFQGAWLGCPEPWEPLLDGGACPQAHTVGTGETPLHLAARFSRPTAARRLLEAGANPNQPDRAGRTPLHAAVAADAREVCQLLLRSRQTAVDARTEDGTTPLMLAARLAVEDLVEELIAAQADVGARDKWGKTALHWAAAVNNARAARSLLQAGADKDAQDNREQTPLFLAAREGAVEVAQLLLGLGAARELRDQAGLAPADVAHQRNHWDLLTLLEGAGPPEARHKATPGREAGPFPRARTVSVSVPPHGGGALPRCRTLSAGAGPRGGGACLQARTWSVDLAARGGGAYSHCRSLSGVGAGGGPTPRGRRFSAGMRGPRPNPAIMRGRYGVAAGRGGRVSTDDWPCDWVALGACGSASNIPIPPPCLTPSPERGSPQLDCGPPALQEMPINQGGEGKK

SEQ ID NO: 5 - protein, 1218 aa, Homo sapiens [sequence omitted]

SEQ ID NO: 6 - protein, 685 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 7 - protein, 1238 aa, Homo sapiens [sequence omitted]

SEQ ID NO: 8 - protein, 723 aa, Homo sapiens
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMTCADGPCFNGGRCSDSPDGGYSCRCPVGYSGFNCEKKIDYCSSSPCSNGAKCVDLGDAYLCRCQAGFSGRHCDDNVDDCASSPCANGGTCRDGVNDFSCTCPPGYTGRNCSAPVSRCEHAPCHNGATCHERGHRYVCECARGYGGPNCQFLLPELPPGPAVVDLTEKLEGQGGPFPWVAVCAGVILVLMLLLGCAAVVVCVRLRLQKHRPPADPCRGETETMNNLANCQREKDISVSIIGATQIKNTNKKADFHGDHSADKNGFKARYPAVDYNLVQDLKGDDTAVRDAHSKRDTKCQPQGSSGEEKGTPTTLRGGEASERKRPDSGCSTSKDTKYQSVYVISEEKDECVIATEV

SEQ ID NO: 9 - protein, 850 aa, Homo sapiens
MTSHYVIAIFALMSSCLATAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTVMGIAFAAFVIGALLTGALWYIYSHTGETAGRQQVPTSPPASENSSAAHSIGSTQSTPCSSSSTA

SEQ ID NO: 10 - protein, 454 aa, Homo sapiens
FVNDSVTKSIVALRLTLVVKVSTCVPGESHANDLECSGKGKCTTKPSEATFSCTCEEQYVGTFCEEYDACQRKPCQNNASCIDANEKQDGSNFTCVCLPGYTGELCQSKIDYCILDPCRNGATCISSLSGFTCQCPEGYFGSACEEKVDPCASSPCQNNGTCYVDGVHFTCNCSPGFTGPTCAQLIDFCALSPCAHGTCRSVGTSYKCLCDPGYHGLYCEEEYNECLSAPCLNAATCRDLVNGYECVCLAEYKGTHCELYKDPCANVSCLNGATCDSDGLNGTCICAPGFTGEECDIDINECDSNPCHHGGSCLDQPNGYNCHCPHGWVGANCEIHLQWKSGHMAESLTNMPRHSLYIIIGALCVAFILMLIILIVGICRISRIEYQGSSRPAYEEFYNCRSIDSEFSNAIASIRHARFGKKSRPAMYDVSPIAYEDYSPDDKPLVTLIKTKDL

SEQ ID NO: 11 - protein, 536 aa, Homo sapiens
MALTSFLPAPTQLSQDQLEAEEKARSQRSRQTSLVSSRREPPPYGYRKGWIPRLLEDFGDGGAFPEIHVAQYPLDMGRKKKMSNALAIQVDSEGKIKYDAIARQGQSKDKVIYSKYTDLVPKEVMNADDPDLQRPDEEAIKEITEKTRVALEKSVSQKVAAAMPVRAADKLAPAQYIRYTPSQQGVAFNSGAKQRVIRMVEMQKDPMEPPRFKINKKIPRGPPSPPAPVMHSPSRKMTVKEQQEWKIPPCISNWKNAKGYTIPLDKRLAADGRGLQTVHINENFAKLAEALYIADRKAREAVEMRAQVERKMAQKEKEKHEEKLREMAQKARERRAGIKTHVEKEDGEARERDEIRHDRRKERQHDRNLSRAAPDKRSKLQRNENRDISEVIALGVPNPRTSNEVQYDQRLFNQSKGMDSGFAGGEDEIYNVYDQAWRGGKDMAQSIYRPSKNLDKDMYGDDLEARIKTNRFVPDKEFSGSDRRQRGREGPVQFEEDPFGLDKFLEEAKQHGGSKRPSDSSRPKEHEHEGKKRRKE

SEQ ID NO: 12 - protein, 500 aa, Homo sapiens
MDHTEGSPAEEPPAHAPSPGKFGERPPPKRLTREAMRNYLKERGDQTVLILHAKVAQKSYGNEKRFFCPPPCVYLMGSGWKKKKEQMERDGCSEQESQPCAFIGIGNSDQEMQQLNLEGKNYCTAKTLYISDSDKRKHFMLSVKMFYGNSDDIGVFLSKRIKVISKPSKKKQSLKNADLCIASGTKVALFNRLRSQTVSTRYLHVEGGNFHASSQQWGAFFIHLLDDDESEGEEFTVRDGYIHYGQTVKLVCSVTGMALPRLIIRKVDKQTALLDADDPVSQLHKCAFYLKDTERMYLCLSQERIIQFQATPCPKEPNKEMINDGASWTIISTDKAEYTFYEGMGPVLAPVTPVPVVESLQLNGGGDVAMLELTGQNFTPNLRVWFGDVEAETMYRCGESMLCVVPDISAFREGWRWVRQPVQVPVTLVRNDGIIYSTSLTFTYTPEPGPRPHCSAAGAILRANSSQVPPNESNTNSEGSYTNASTNSTSVTSSTATVVS

SEQ ID NO: 13 - protein, 166 aa, Homo sapiens
MAPSTVAVELLSPKEKNRLRKPVVEKMRRDRINSSIEQLKLLLEQEFARHQPNSKLEKADILEMAVSYLKHSKAFVAAAGPKSLHQDYSEGYSWCLQEAVQFLTLHAASDTQMKLLYHFQRPPAAPAAPAKEPKAPGAAPPPALSAKATAAAAAAHQPACGLWRPW

SEQ ID NO: 14 - protein, 230 aa, Homo sapiens
MVTRDRAENRDGPKMLKPLVEKRRRDRINRSLEELRLLLLERTRDQNLRNPKLEKAEILEFAVGYLRERSRVEPPAAAAPGVPRSPVQDAEALASCYLSGFRECLLRLAAFAHDASPAARAQLFSALHGYLRPKPPRPKPVDPRPPAPRPSLDPAAPALGPALHQRPPVHQGHPSPRCAWSPSLCSPRAGDSGAPAPLTGLLPPPPPPHRQDGAPKAPLPPPPAFWRPWP

SEQ ID NO: 15 - protein, 426 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 16 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 17 - DNA, 22 bp, Artificial
  Synthetic Primer
ggcagaccgagatgaatcctca

SEQ ID NO: 18 - DNA, 21 bp, Artificial
  Synthetic Primer
caggtccaggggtcttggtcc

SEQ ID NO: 19 - DNA, 20 bp, Artificial
  Synthetic Primer
ctgcaagatacatggctcca

SEQ ID NO: 20 - DNA, 21 bp, Artificial
  Synthetic Primer
ctcgatctctcaacacgttgt

SEQ ID NO: 21 - protein, 486 aa, Homo sapiens
MAWIKRKFGERPPPKRLTREAMRNYLKERGDQTVLILHAKVAQKSYGNEKRFFCPPPCVYLMGSGWKKKKEQMERDGCSEQESQPCAFIGIGNSDQEMQQLNLEGKNYCTAKTLYISDSDKRKHFMLSVKMFYGNSDDIGVFLSKRIKVISKPSKKKQSLKNADLCIASGTKVALFNRLRSQTVSTRYLHVEGGNFHASSQQWGAFFIHLLDDDESEGEEFTVRDGYIHYGQTVKLVCSVTGMALPRLIIRKVDKQTALLDADDPVSQLHKCAFYLKDTERMYLCLSQERIIQFQATPCPKEPNKEMINDGASWTIISTDKAEYTFYEGMGPVLAPVTPVPVVESLQLNGGGDVAMLELTGQNFTPNLRVWFGDVEAETMYRCGESMLCVVPDISAFREGWRWVRQPVQVPVTLVRNDGIIYSTSLTFTYTPEPGPRPHCSAAGAILRANSSQVPPNESNTNSEGSYTNASTNSTSVTSSTATVVS

SEQ ID NO: 22 - protein, 487 aa, Homo sapiens
MAPVVTGKFGERPPPKRLTREAMRNYLKERGDQTVLILHAKVAQKSYGNEKRFFCPPPCVYLMGSGWKKKKEQMERDGCSEQESQPCAFIGIGNSDQEMQQLNLEGKNYCTAKTLYISDSDKRKHFMLSVKMFYGNSDDIGVFLSKRIKVISKPSKKKQSLKNADLCIASGTKVALFNRLRSQTVSTRYLHVEGGNFHASSQQWGAFFIHLLDDDESEGEEFTVRDGYIHYGQTVKLVCSVTGMALPRLIIRKVDKQTALLDADDPVSQLHKCAFYLKDTERMYLCLSQERIIQFQATPCPKEPNKEMINDGASWTIISTDKAEYTFYEGMGPVLAPVTPVPVVESLQLNGGGDVAMLELTGQNFTPNLRVWFGDVEAETMYRCGESMLCVVPDISAFREGWRWVRQPVQVPVTLVRNDGIIYSTSLTFTYTPEPGPRPHCSAAGAILRANSSQVPPNESNTNSEGSYTNASTNSTSVTSSTATVVS

SEQ ID NO: 23 - protein, 485 aa, Homo sapiens
MGGCRKFGERPPPKRLTREAMRNYLKERGDQTVLILHAKVAQKSYGNEKRFFCPPPCVYLMGSGWKKKKEQMERDGCSEQESQPCAFIGIGNSDQEMQQLNLEGKNYCTAKTLYISDSDKRKHFMLSVKMFYGNSDDIGVFLSKRIKVISKPSKKKQSLKNADLCIASGTKVALFNRLRSQTVSTRYLHVEGGNFHASSQQWGAFFIHLLDDDESEGEEFTVRDGYIHYGQTVKLVCSVTGMALPRLIIRKVDKQTALLDADDPVSQLHKCAFYLKDTERMYLCLSQERIIQFQATPCPKEPNKEMINDGASWTIISTDKAEYTFYEGMGPVLAPVTPVPVVESLQLNGGGDVAMLELTGQNFTPNLRVWFGDVEAETMYRCGESMLCVVPDISAFREGWRWVRQPVQVPVTLVRNDGIIYSTSLTFTYTPEPGPRPHCSAAGAILRANSSQVPPNESNTNSEGSYTNASTNSTSVTSSTATVVS

(19) United States
(12) Patent Application Publication
(10) Pub. No.: US 2015/0140043 A1
(43) Pub. Date: May 21, 2015

(54) IMMUNOMODULATORY METHODS USING NOTCH AGONISTS

(71) Applicants: INSTITUT NATIONAL DE LA SANTE DE LA RECHERCHE MEDICAL (INSERM), Paris (FR); UNIVERSITE PARIS EST CRETEIL VAL DE MARNE, Creteil (FR); ASSISTANCE PUBLIQUE-HOPITAUX DE PARIS, Paris (FR)

(72) Inventors: Yves Levy, Creteil (FR); Sophie Hüe, Creteil (FR)

(21) Appl. No.: 14/399,977

(22) PCT Filed: May 7, 2013
(86) PCT No.: PCT/EP2013/059534

§ 371 Date: Nov. 10, 2014

(30) Foreign Application Priority Data
May 10, 2012 (EP) 12305521.2

(51) Int. Cl. A61K 39/00; G01N 33/50; C12Q 1/68; A61K 45/06; G01N 33/74

(52) U.S. Cl. CPC A61K 39/0008; A61K 45/06; G01N 33/74; C12Q 1/6883; G01N 33/505; C12Q 2600/158; C12Q 2600/106; G01N 2800/52; G01N 2800/24
USPC 424/278.1; 435/375; 435/7.24; 435/6.12; 435/6.11; 506/9; 435/29

(57) ABSTRACT

The invention relates to materials and methods for regulating immune pathways using NOTCH and NOTCH agonists. In particular, said agonists may be used in methods for sensitizing CD4+ CD25− cells to CD4+ CD25+ cell-mediated immunosuppression, for enhancing the TGF-β response of CD4+ CD25− cells.

### FIELD OF THE INVENTION

The invention relates to materials and methods for regulating immune pathways using NOTCH and NOTCH agonists. In particular, said agonists may be used in methods for sensitizing CD4+ CD25− cells to CD4+ CD25+ cell-mediated immunosuppression, for enhancing the TGF-β response of CD4+ CD25− cells.

### BACKGROUND OF THE INVENTION

The NOTCH signalling system is conserved from Drosophila to humans and regulates cell differentiation, proliferation and survival. NOTCH pathways play an important role in embryonic development, T cell development and function, and in disease processes including carcinogenesis and autoimmunity. In mammals there are four NOTCH receptors and five NOTCH ligands (Jagged-1, Jagged-2, Delta-like 1 [DL-1] and DL-4) (Yuan, J. S. et al., 2010. Annu Rev Immunol 28:343).

NOTCH proteins exert their pleiotropic effects through the regulation of expression of various downstream genes, many of which require the interaction of NOTCH proteins with the DNA binding transcription factor CSL in order to form a short-lived nuclear transcription complex (Osborne, B. A. et al., 2007; Nat Rev Immunol 7:64). After engagement with its ligands, successive proteolytic events cause clipping of the NOTCH protein. The first is mediated by ADAM proteases and the second by the γ-secretase complex, in which presenilins (PS1 and PS2) constitute the active center of the enzyme complex. These proteolytic events ultimately release the intracellular domain of NOTCH (NICD). The formation of a complex of activated intracellular NOTCH protein and CSL converts CSL from a transcriptional repressor to a transcriptional transactivator. The genes encoding the HES (hairy and enhancer of split) family of basic helix-loop-helix proteins are NOTCH targets that are known to be essential for T-cell development and signalling.

In addition to influencing Th1 and Th2-cell differentiation (Amsen, D., A. et al., 2009, Nat Rev Immunol 9:116), NOTCH signalling has been also involved in the differentiation and expansion of regulatory T cells (Treg) (Ostroukhova, M., Z. et al., 2006, J Clin Invest 116:996). Several reports have shown that the presence of NOTCH ligands, mostly of the Jagged family, can enhance Treg cell differentiation and function in vitro (Vigouroux, S., E. et al., 2003, J Virol 77:10872). For example, exposure of Treg cells to Jagged-2 expressed by hematopoietic progenitor cells has been shown to modulate peripheral Treg expansion and prevent the development of diabetes in an autoimmune disease mice model (Kared, H., H. et al., 2006, Immunity 25:823). Transgenic mice over expressing the active intra-cellular form of NOTCH3 exhibit an increased percentage of Treg and are refractory to the induction of experimental autoimmune diabetes when treated with streptozotocin (Anastasi, E., et al., 2003, J Immunol 171:4504).

Moreover, there is emerging data suggesting that NOTCH can crosstalk or cooperate with other signalling pathways and thereby broaden the spectrum of target genes that are influenced by NOTCH signalling. For example, the interaction of NOTCH and TGF-β signal pathways plays a role in Treg effector function through a modulation of FoxP3 expression or by facilitating TGF-β mediated suppressive function of Tregs (Samon, J. B. et al., 2008, Blood 112:1813, Asano, N. et al., 2008. J Immunol 180:2796).

T helper 17 (Th17) cells are an important inflammatory component and have been shown to play a role in antimicrobial immunity and to promote inflammation in a number of autoimmune diseases (Stockinger, B., and M. Veldhoen. 2007. Curr Opin Immunol 19:281). The factors determining the differentiation of this subset have been clearly delineated in mice. TGF-β and IL-6 are dominant in directing the differentiation of Th17 cells from naïve CD4⁺ T cells and the transcription factor RORγT is an essential component in this process (Ivanov, I I, B. S. et al., 2006. Cell 126:1121). Human Th17 cells were initially shown to develop in the absence of TGF-β, requiring only IL-6 and IL-1β or IL-23 and IL-1β. Later studies refuted these observations, demonstrating an essential role of TGF-β in the differentiation of naïve human CD4⁺ T cells toward the Th17 lineage or for their maintenance (Gutcher, I. et al., 2011 Immunity 34:396-16).

Most of the reports published to date have focused on the influence of NOTCH signalling on Treg cells. The literature does not disclose any function of NOTCH signalling in modulation of effector T cell response to Treg-mediated suppressive effects.

Moreover, the resistance of T cells to Treg mediated immunosuppresion has been involved in immune diseases pathogenesis notably Juvenile idiopathic arthritis (JIA), underlying the essential role of Treg mediated immunosuppression in human, autoimmune disease (Wehrens E J. et al., Blood. 2011 Sep. 29; 118(13):3538-48; Haufe S. et al., Arthritis Rheum. 2011 October; 63(10):3153-62. doi: 10.1002/art.30503).

### SUMMARY OF THE INVENTION

The inventors have shown that NOTCH activation through its ligands, increases exquisitely the sensitivity of effector CD4⁺CD25⁻ T cells to the suppressive effect of Treg CD4⁺CD25⁺ cells even at low frequency. This effect is mediated through an upregulation of TGF-βRII and the phosphorylated form of Smad 3 protein on effector T cells. They have just for the first time, demonstrated the effect of NOTCH signalling on effector CD4⁺CD25⁻ T cells. Moreover, the inventors have demonstrated that HES-1, the best known intracellular target of NOTCH, transactivates the TGF-βRII promoter.

In one aspect, therefore, the invention provides a NOTCH agonist for use in a method for sensitizing CD4+ CD25− cells to CD4+ CD25+ cell-mediated immunosuppression or for enhancing the TGF-β response of CD4+ CD25− cells.

The invention further provides a NOTCH agonist for use in a method of prevention and/or treatment of an immune dysregulatory disorder of an individual having CD4+ CD25− cells which are resistant to CD4+ CD25+ cell-mediated immunosuppression; and/or which underexpress TGF-β receptor.

The invention further provides a NOTCH agonist for use in an immunotherapy of an individual having CD4+ CD25− cells which are resistant to CD4+ CD25+ cell-mediated immunosuppression; and/or which underexpress TGF-β receptor.

The invention further provides a NOTCH agonist for use in inducing immunosuppression in an individual having CD4+ CD25− cells which are resistant to CD4+ CD25+ cell-mediated immunosuppression; and/or which under expressed TGF-β receptor. Typically, the invention further provides a NOTCH agonist for use in reducing the immune response to an allergen or antigenic determinant thereof.

In some embodiments, said method is a method for enhancing the expression of the TGF-β receptor in CD4+ CD25− cells, for example a type II TGF-β receptor.

Said method may be a method of prevention and/or treatment of an immune dysregulatory disorder, such as an autoimmune disease, inflammatory disorder, cardio-vascular disease or diabetes.

In some embodiments said method is a method of prevention of organ or tissue transplant rejection.

In some embodiments, said NOTCH agonist is selected from the group consisting of Delta-like 4, Jagged-1 and a biologically active fragment thereof.

In some embodiments, the NOTCH agonist is administered in combination with a product selected from the group consisting of anti-diabetes and immunomodulatory drugs, either simultaneously, separately or sequentially.

Preferably, said immunomodulatory drug is selected from the group consisting of anti-histamine and anti-inflammatory drugs.

The invention also provides a method for sensitizing CD4+ CD25− cells to CD4+ CD25+ cell-mediated immunosuppression, or for enhancing the TGF-β response of CD4+CD25− cells, comprising administering to an individual a NOTCH agonist.

The invention further provides a method for sensitizing CD4+ CD25− cells to CD4+ CD25+ cell-mediated immunosuppression or enhancing the TGF-β response of CD4+ CD25− cells comprising the steps of:

a) providing a biological sample comprising CD4+ CD25− cells

b) contacting the biological sample with a NOTCH agonist.

Preferably, a said method is an in vitro or ex vivo method.

According to the invention, “sensitizing CD4+ CD25− cells to CD4+ CD25+ cell-mediated immunosuppression” means to induce or confer sensitivity to a CD4+ CD25-cell which is resistant to CD4+ CD25+ cell-mediated immunosuppression. A CD4+ CD25− cell sensitive to CD4+ CD25+ cell-mediated immunosuppression refers to the phenotype of a CD4+ CD25− cell which responds to immunosuppression stimuli mediated by CD4+ CD25+ cells. Such phenotype may be proliferation of CD4+ CD25− cells when co-cultured with CD4+ CD25+ cells . . . .

According to the invention, “enhancing the TGF-β response of CD4+ CD25− cells” means to induce or improve sensitivity of a CD4+ CD25− cell which is resistant or not much sensitive to a TGF-β stimulation. A CD4+ CD25− cell sensitive to a TGF-β stimulation refers to the phenotype of a CD4+ CD25− cell which responds to TGF-β stimuli notably mediated by CD4+ CD25+ cells. Such phenotype may be proliferation of CD4+ CD25− cells when co-cultured with CD4+ CD25+ cells or in the presence of a TGF-β stimulation and comparing the measurements obtained with a control. Such phenotype may further be a positive immunostaining for TGF-β receptor or an increase in the immunostaining for TGF-β receptor comparing with a control.

Also provided is a method of selecting a patient suffering from an immune dysregulatory disorder to be treated by a NOTCH agonist which method comprises the steps of:

a) providing a biological sample comprising CD4+ CD25− cells from said patient;

b) determining whether:
said CD4+ CD25− cells are resistant to CD4+ CD25+ cell-mediated immunosuppression; and/or
TGF-β receptor is expressed on said CD4+ CD25− cells; and

c) selecting said patient for treatment by a NOTCH agonist if said CD4+ CD25− cells are CD4+ CD25+ cell resistant, and/or if TGF-β receptor is expressed at a significantly lower level in said biological sample than in a control sample. Preferably, a said method is an in vitro or ex vivo method.

According to the invention, “CD4+ CD25− cells resistant to CD4+ CD25+ cell-mediated immunosuppression” refers to the phenotype of a CD4+ CD25− cell which does not respond to immunosuppression stimuli mediated by CD4+ CD25+ cells compared with a sensitive CD4+ CD25− cell. Such phenotype may be the non-proliferation of CD4+ CD25-cells when co-cultured with CD4+ CD25+ cells or in the presence of TGF-β stimulation. For example, said determination of whether CD4+ CD25− cells are resistant to CD4+ CD25+ cell-mediated immunosuppression may be performed by measuring CD4+ CD25− T cell proliferation when co-cultured with CD4+ CD25+ cells or in the presence of a TGF-β stimulation and comparing the measurements obtained with a control.

In some embodiments, said NOTCH agonist is selected from the group consisting of Delta-like 1, Delta-like 4, Jagged-1 and a biologically active fragment thereof.

As used herein, the term “NOTCH” encompasses any naturally occurring isoform of the NOTCH protein, including the protein of SEQ ID NO: 1, allelic variants thereof, splice variants thereof and homologous proteins in other species. Preferably, by NOTCH is meant any one of mammalian (preferably human) NOTCH 1 (ACC. NO AAG33848.1 sequence SEQ ID NO: 1), NOTCH 2 (ACC. NO AAA36377.2, sequence SEQ ID NO: 2), NOTCH 3 (ACC. NO AAC15789.1, sequence SEQ ID NO: 3) or NOTCH 4 (ACC. NO Q99466.2, sequence SEQ ID NO: 4). Most preferably, NOTCH has the sequence of SEQ ID NO: 1.

As used herein, the term “NOTCH agonist” refers to a compound that induces or activates NOTCH biological activity. The biological activity of NOTCH depends on the amount of the protein (i.e. its expression level) as well as on the activity of the protein. Therefore, the NOTCH agonist may activate or induce either NOTCH expression, or NOTCH protein activity. Most preferably, NOTCH agonist is NOTCH 1 agonist.

The agonists according to the present invention include those which specifically bind to NOTCH thereby improving or inducing signal transduction. Such agonists may include naturally occurring ligands of NOTCH, such as JAG1 (ACC. NO AAC51731.1; GI:2228793) of sequence SEQ ID NO: 5, JAG2 (ACC. NO AAB61285.1; GI:2197067) of sequence SEQ ID NO: 7; DL-1 (ACC. NO AAQ89251.1; GI:37182902) of sequence SEQ ID NO: 8; DL-4 (ACC. NO AAQ89253.1; GI:37182906) of sequence SEQ ID NO: 6, Delta/Notch-like EGF-related receptor (DNER) (ACC. NO AAH24766.2; GI:34783189) of sequence SEQ ID NO: 10; Ski-interacting protein (SKIP) (ACC. NO AAV38864.1; GI:54696984) of sequence SEQ ID NO: 11; CSL (CBF1 Suppressor of Hairless Lag-1; UniProt accession no Q06330) isoforms 1 to 4 having respectively sequences SEQ ID NO: 12 (ACC. NO NP_{—}005340.2; GI:42560227), SEQ ID NO: 22 (ACC. NO NP_{—}056958.3; GI:42560229); SEQ ID NO: 23 (ACC. NO NP_{—}976028.1; GI:42560225) and SEQ ID NO: 21 (ACC. NO NP_{—}976029.1; GI:42560223); HES-5 (ACC. NO NP_{—}001010926.1; GI:58219048) of sequence SEQ ID NO: 13 or HES-7 (ACC. NO NP_{—}001159439.1; GI:260166650; HES-7 isoform 1) of sequence SEQ ID NO: 14.

In some embodiments an agonist is delta 4 or jagged 1 or a biologically active fragment thereof.

The NOTCH agonist may correspond to any type of molecule, such as e.g. a nucleic acid selected from the group consisting of a chemical molecule (e.g. a small molecule), a peptide preferably a fragment of a NOTCH ligand or its peptidomimetic, a dominant activated mutant of NOTCH or a fragment or a peptidomimetic thereof.

In another embodiment, the agonist for use according to invention is a chemical molecule (preferably a small molecule) that specifically binds to the NOTCH protein.

The agonist for use according to invention may also be an antibody that specifically binds to the NOTCH protein.

A “biologically active fragment” of delta 4 or jagged 1 is a fragment that specifically binds to NOTCH and that activates the same NOTCH downstream signalling pathway as full-length delta 4 or jagged 1. A biologically active fragment may allow sensitizing of CD4+ CD25− cells to CD4+ CD25+ cell-mediated immunosuppression or enhancing the TGF-β response of CD4+ CD25− cells.

Methods for determining whether a compound is a NOTCH agonist are well-known by the person skilled in the art.

For example, the person skilled in the art can assess whether a compound induces NOTCH expression by Western Blotting or by RT-PCR. NOTCH signaling is initiated upon ligand receptor interaction, which results in the proteolytic release of the NOTCH intracellular cytoplasmic domain (NICD). Consequently, the person skilled in the art can further assess whether a compound activates NOTCH biological activity by detecting or measuring a change in the amount or pattern of NOTCH cleavage products.

The biological activity of a NOTCH protein can be assessed through measuring one of the phenomenon in which NOTCH is known to play a role. For instance, NOTCH is known to play a role in transcription, differentiation processes and (tissue) lineage decisions in fetal and postnatal development, etc. By way of example, NOTCH1 and NOTCH2 act as activators of HES transcription (Hairy-Enhancer of split encoding genes) among which HES-1, HES-5, and HES-7 are known targets of the NOTCH receptor. The inventors have demonstrated that NOTCH receptor is implicated in TGF-β receptor type II activation. A compound inducing or improving the capacity of NOTCH to play a role in one of these phenomena is defined as a NOTCH agonist.

For example, determining whether a compound is a NOTCH agonist can be done by assessing the level of a protein or transcript whose expression is regulated by NOTCH in the presence and in the absence of a candidate compound such as HES-1, HES-5, HES-7 or TGF-β receptor. A preferred example is TGF-β receptor more particularly TGF-β receptor type II. A compound enhancing or improving level of a protein or transcript whose expression is regulated by NOTCH is defined as a NOTCH agonist.

NOTCH functions as a receptor, and mammals have four NOTCH receptors (NOTCH1, NOTCH2, NOTCH3 and NOTCH4) and many ligands, including jagged 1 (JAG1) and JAG2 (homologues of serrate), and delta-like proteins (DL-1, DL-4) Delta/NOTCH-like EGF-related receptor (DNER). NOTCH and its ligands are single-pass transmembrane heterodimers.

The biological activity of NOTCH may also be measured by assessing the capacity of NOTCH to bind to its natural binding partners such as e.g. JAG1, JAG2, DL-1, DL-4 and DNER or the capacity of NICD domain of NOTCH to bind to CSL, SKIP proteins. The binding of NOTCH to JAG1, JAG2, DL-1, DL-4 or DNER may for example be assessed using a co-immunoprecipitation assay, a pull-down assay or the yeast two hybrid system (Y2H). A compound that improves binding of NOTCH to JAG1 of sequence SEQ ID NO: 5, JAG2 of sequence SEQ ID NO: 7, DL-1 of sequence SEQ ID NO: 8, DL-4 of sequence SEQ ID NO: 6, DNER of sequence SEQ ID NO: 10, HES-5 of sequence SEQ ID NO: 13 or HES-7 of sequence SEQ ID NO: 14 is defined as a NOTCH agonist.

Preferably, the NOTCH agonist is capable of specifically binding to NOTCH.

The term “TGF-β” encompasses any naturally occurring isoform of the TGF-β protein including, but not limited to, TGFβ1, TGFβ2, TGFβ3, TGFβ4 and TGFβ5, allelic variants thereof, splice variants thereof and homologous proteins in other species. Preferably, TGF-β is a mammalian protein (preferably human).

“TGF-β receptor” or “TGF-β R” means TGF-β receptor, for example a TGF-β receptor type I of sequence SEQ ID NO:15 (ACC. NO AAH71181), TGF-β receptor type II of sequence SEQ ID NO: 16 (ACC. NO ABG65632.1) or TGF-β receptor type III of sequence SEQ ID NO: 9 (ACC. NO CAI22637.1). As used herein, TGF-β receptor refers to a serine/threonine kinase receptor that binds a member of the TGF-β family (e.g., TGF-β31, TGF-β2, TGF-β3, etc). Generally, a TGF-βR may exist in several different isoforms, and may be homo- or heterodimeric. Three TGF-β receptor types may be distinguished by their structural and functional properties. Receptor types I and II (TGF-βRI and TGF-βRII, respectively) have a high affinity for TGF-β1 and low affinity for TGF-β2. TGF-β receptor type III has a high affinity for both TGF-β1 and TGF-β2.

The term “variants” includes protein and nucleic acid variants. Variant proteins may be naturally occurring variants, such as splice variants, alleles and isoforms, or they may be produced by recombinant means. Variations in amino acid sequence may be introduced by substitution, deletion or insertion of one or more codons into the nucleic acid sequence encoding the protein that results in a change in the amino acid sequence of the protein. Optionally the variation is by substitution of 1, 2, 3, 4, 5, 6, 7, 8, 9, 10, 11, 12, 13, 14, 15, 16, 17, 18, 19, 20 or more amino acids with any other amino acid in the protein. Amino acid substitutions may be conservative or non-conservative. Preferably, substitutions are conservative substitutions, in which one amino acid is substituted for another amino acid with similar structural and/or chemical properties. Additionally or alternatively, the variation may be by addition or deletion of 1, 2, 3, 4, 5, 6, 7, 8, 9, 10, 11, 12, 13, 14, 15, 16, 17, 18, 19, 20 or more amino acids within the protein.

Amino acid substitutions may be conservative or non-conservative. Preferably, substitutions are conservative substitutions, in which one amino acid is substituted for another amino acid with similar structural and/or chemical properties. Exemplary conservative substitutions are listed below.

Ala (A) val; leu; ile

Arg (R) lys; gin; asn

Asn (N) gln; his; lys

Asp (D) glu

Cys (C) ser

Gln (Q) asn

Glu (E) asp

Gly (G) pro; ala

His (H) asn; Gln; lys; arg

He (I) leu; val; met; ala

norleucine leu

Leu (L) norleucine; ile; met; ala; phe

Lys (K) arg; Gln; asn

Met (M) leu; phe; ile

Phe (F) leu; val; ile; ala; tyr

Pro (P) ala

Ser (S) thr

Thr (T) ser

Trp (W) tyr; phe

Tyr (Y) trp; phe; thr; ser

Val (V) ile; leu; met; phe; ala; norleucine

Variant proteins may include proteins that have at least about 80% amino acid sequence identity with a polypeptide sequence disclosed herein. Preferably, a variant protein will have at least about 80%, 81%, 82%, 83%, 84%, 85%, 86%, 87%, 88%, 89%, 90%, 91%, 92%, 93%, 94%, 95%, 96%, 97%, 98%, 99% amino acid sequence identity to a full-length polypeptide sequence or a fragment of a polypeptide sequence as disclosed herein. Amino acid sequence identity is defined as the percentage of amino acid residues in the variant sequence that are identical with the amino acid residues in the reference sequence, after aligning the sequences and introducing gaps, if necessary, to achieve the maximum percent sequence identity, and not considering any conservative substitutions as part of the sequence identity. Sequence identity may be determined over the full length of the variant sequence, the full length of the reference sequence, or both. Methods for sequence alignment and determination of sequence identity are well known in the art, for example using publicly available computer software such as BioPerl, BLAST, BLAST-2, CS-BLAST, FASTA, ALIGN, ALIGN-2, LALIGN, Jaligner, matcher or Megalign (DNASTAR) software and alignment algorithms such as the Needleman-Wunsch and Smith-Waterman algorithms.

For example, the percentage identity may be calculated by performing a pairwise global alignment based on the Needleman-Wunsch alignment algorithm to find the optimum alignment (including gaps) of two sequences along their entire length, for instance using Needle, and using the BLOSUM62 matrix with a gap opening penalty of 10 and a gap extension penalty of 0.5.

Fragments of the proteins and variant proteins disclosed herein are also encompassed by the invention. Such fragments may be truncated at the N-terminus or C-terminus, or may lack internal residues, for example, when compared with a full length protein. Certain fragments lack amino acid residues that are not essential for enzymatic activity. Preferably, said fragments are at least about 10, 20, 30, 40, 50, 60, 70, 80, 90, 100, 110, 120, 150, 250, 300, 350, 400, 450, 500 or more amino acids in length.

As used herein the term “polypeptide” refers to any chain of amino acids linked by peptide bonds, regardless of length or post-translational modification. Polypeptides include natural proteins, synthetic or recombinant polypeptides and peptides (i.e. polypeptides of less than 50 amino acids) as well as hybrid, post-translationally modified polypeptides, and peptidomimetic.

As used herein, the term “amino acid” refers to the 20 standard alpha-amino acids as well as naturally occurring and synthetic derivatives. A polypeptide may contain L or D amino acids or a combination thereof.

The term “small molecule” refers to a molecule of less than 1,000 daltons, in particular organic or inorganic compounds. Structural design in chemistry should help to find such a molecule. The molecule may have been identified by a screening method disclosed in the present invention.

As used herein, the term “specifically binding” has its usual meaning in the art. For instance, a molecule that specifically binds to a polypeptide can be defined as a molecule that is capable of competing with a known ligand of said polypeptide in a competitive binding assay. In the context of the present invention, the specific binding is preferably a selective binding, i.e. the molecule has a tendency to bind to a very limited number of binding partners.

A “binding partner” refers to a molecule (peptidyl or non-peptidyl) that interacts directly with a target protein (such as TGF-β receptor) and capable of specifically binds to a target protein, optionally said binding partner may neutralizing, blocking, inhibiting, abrogating, reducing or interfering with said target protein activities including its binding to one or more other cellular partners. Said binding partner is a protein partner or a fusion protein partner or its binding domain or peptidomimetic or an immunoglobulin.

Methods for determining whether a polypeptide, a protein or a chemical molecule is capable of specifically binding to NOTCH are well-known to the skilled in the art. Such methods for example include dose response assays with a competitive ligand, co-immunoprecipitation, surface plasmon resonance (e.g. using a BIACore) and yeast double-hybrid assays. Herein, the term “specific binding” to a protein has its usual meaning in the art, and is used to qualify a binding as opposed to a “non-specific binding”.

Said binding partner may be marked to be easily detected by the skilled person in the art. Binding partner binding to a protein of interest may be detected through the use of chemical reagents that generate a detectable signal. In one method, the binding can be detected through the use of a binding partner that is conjugated to a labeled polymer. Examples of labeled polymers include but are not limited to polymer-enzyme conjugates. The enzymes in these complexes are typically used to catalyze the deposition of a chromogen at the antigen-antibody binding site, thereby resulting in cell staining that corresponds to expression level of the biomarker of interest. Enzymes of particular interest include horseradish peroxidase (HRP) and alkaline phosphatase (AP). Said binding partner may be conjugated to fluorophore or may be tagged with for example a fluorescent maker such as fluorescent protein domain. Samples may be examined via automated microscopy or by personnel with the assistance of computer software that facilitates the identification of positive staining cells.

In some embodiment, said specific binding partner binds which specifically to the gene sequence of said target protein or to its complementary sequence may be a sense primer and/or an antisense primer

As used herein the term “peptidomimetic” refers to peptide-like structures which have non-amino acid structures substituted but which mimic the chemical structure of a peptide and retain the functional properties of the peptide. Peptidomimetics may be designed in order to increase peptide stability, bioavailability, solubility, etc.

In some embodiments, an NOTCH agonist may be a biologically active peptidomimetic, preferably, a peptidomimetic that specifically binds to NOTCH and is susceptible to activate the same NOTCH downstream signalling pathway as delta 4 or jagged 1. Said biologically active peptidomimetic may allow sensitizing CD4+ CD25− cells to CD4+ CD25+ cell-mediated immunosuppression or enhancing the TGF-β response of CD4+ CD25− cells.

In the context of the present invention, the individual or patient preferably is a human individual. However, the veterinary use of the NOTCH agonist according to the present invention is also envisioned. The individual may thus also correspond to a non-human individual, preferably a non-human mammal such as a rodent, a feline, a canine, or a primate.

“Treatment” includes both therapeutic treatment and prophylactic or preventative treatment, wherein the object is to prevent or slow down the targeted pathologic condition or disorder. Those in need of treatment include those already with the disorder as well as those prone to have the disorder or those in whom the disorder is to be prevented. The terms ‘therapy’, ‘therapeutic’, ‘treatment’ or ‘treating’ include reducing, alleviating or inhibiting or eliminating the symptoms or progress of a disease, as well as treatment intended to reduce, alleviate, inhibit or eliminate said symptoms or progress. Desirable effects of treatment include preventing occurrence or recurrence of disease, alleviation of symptoms, diminishment of any direct or indirect pathological consequences of the disease, preventing immune dysregulation, decreasing the rate of disease progression, amelioration or palliation of the disease state, and remission or improved prognosis. In some embodiments, methods and compositions of the invention are used to delay development of a disease or disorder or to slow the progression of a disease or disorder.

Treatment in accordance with the invention includes a method of treating an immune dysregulatory disorders or other immune over-regulated disorder which comprises administering to a patient in need of treatment a protein, vector or pharmaceutical composition of the invention. Preferably, the treatment further comprises administering to said patient a chemotherapeutic drug, preferably a drug in prodrug form. The two components may be administered together, for example in the form of a combined pill, or separately. Administration may be sequential or simultaneous. ‘Sequential’ administration indicates that the components are administered at different times or time points, which may nonetheless be overlapping. Simultaneous administration indicates that the components are administered at the same time.

Preferably, an effective amount, preferably a therapeutically effective amount of the protein or vector of the invention is administered. An ‘effective amount’ refers to an amount effective, at dosages and for periods of time necessary, to achieve the desired therapeutic or prophylactic result, notably to treat or prevent the immune dysregulatory disorder. The effective amount may vary according to the NOTCH agonist or the vector containing a NOTCH agonist nucleic acid sequence is administered or may vary according to the NOTCH agonist or the vector containing a NOTCH agonist nucleic acid sequence and the immune suppressive drug or prodrug with which is co-administered. The determination of appropriate amounts for any given composition is within the skill in the art, through standard series of tests designed to assess appropriate therapeutic levels.

A “therapeutically effective amount” of a protein or vector of the invention may vary according to factors such as the disease state, age, sex, and weight of the individual, and the ability of the protein, to elicit a desired therapeutic result. A therapeutically effective amount encompasses an amount in which any toxic or detrimental effects of the protein are outweighed by the therapeutically beneficial effects. A therapeutically effective amount also encompasses an amount sufficient to confer benefit, e.g., clinical benefit.

In the case of immune dysregulatory disorders, benign, early or late-stage immune dysregulation, the therapeutically effective amount of the composition of the invention may reduce the number of CD4+ CD25− cells resistant to Treg cell-mediated immunosuppression; reduce the inflammation sites; inhibit (i.e., slow to some extent and preferably stop) effector T cell infiltration into peripheral organs; inhibit (i.e., slow to some extent and preferably stop) inflammation; inhibit or delay, to some extent, inflammation progression; and/or relieve to some extent one or more of the symptoms associated with the disorder. To the extent the drug may prevent resistance to Treg cell-mediated immunosuppression and/or sensitize existing Treg cell-mediated immunosuppression resistant effector T cells, it may enhance the TGF-β response of CD4+ CD25− cells. For immune dysregulatory disorders therapy, efficacy in vivo can, for example, be measured by assessing the duration of survival, time to disease progression (TTP), the response rates (RR), duration of response, and/or quality of life.

The compounds of the invention and notably the NOTCH agonists of the invention may be formulated into pharmaceutical compositions. Solutions said compounds or their pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intraarterial, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, Remington: The Science and Practice of Pharmacy, 21st Edition, Lippincot and Williams, 2005). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compounds of the invention, notably the NOTCH agonists disclosed herein may be formulated in a neutral or salt form. Pharmaceutically-acceptable salts, include the acid addition salts and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

According to the invention, when the method of the invention involves comparing the level of expression of TGF-β receptor or the resistance to CD4+ CD25+ cell-mediated immunosuppression to a predetermined value or a control value obtained from a control sample, the “predetermined value” or the “control value” according to the invention can be a single value such as a level or a mean level of detection of a comportment such as the expression of TGF-β receptor, or the resistance to CD4+ CD25+ cell-mediated immunosuppression as determined in a control sample. The expression “normal sample” or “control sample” refers to a biological sample, preferably a blood sample or a lymphoid tissue sample, of an individual or a reference group of individuals who are not suffering from or who did not develop an immune dysregulatory disorder.

Protein expression may be assessed by using immunologic methods such as detection using polyclonal or monoclonal antibodies. Suitable immunologic methods include enzyme linked immunoassays (ELISA), sandwich, direct, indirect, or competitive ELISA assays, enzyme linked immunospotassays (ELlspot), radio immunoassays (RIA), flow-cytometry assays (FACS), immunohistochemistry, Western Blot, fluorescence resonance energy transfer (FRET) assays, protein chip assays using for example antibodies, antibody fragments, receptor ligands or other agents binding the TGF-β receptors of the invention such as TGF-β.

Level of protein expression of can be performed by other methods which are well known to the person skilled in the art, including in particular quantitative methods involving reverse transcriptase PCR (RT-PCR), such as real-time quantitative RT-PCR (qRT-PCR), and methods involving the use of DNA arrays (macroarrays or microarrays) and In Situ hybridizations.

The term “biological sample” refers to a fluid sample isolated from an individual or from cell culture constituents, as well as samples obtained from, for example, a laboratory procedure. Biological sample refers also to one or more cells, or tissue sample from an individual such as, thymus, lymphoid tissue, plasma, blood or bone marrow sample. “Fluid samples” include, but are not limited to plasma, serum, lymph or whole blood. A biological sample may comprise organelles or membranes isolated from cells, whole cells or tissues, nucleic acid such as genomic DNA in solution or bound to a solid support such as for Southern analysis, RNA in solution or bound to a solid support such as for Northern analysis, cDNA in solution or bound to a solid support, oligonucleotides in solution or bound to a solid support, polypeptides or peptides in solution or bound to a solid support, a tissue, a tissue print and the like.

An “immunomodulatory drug” may be an immunosuppressive agent or an immunostimulating agent.

According to the invention, the term “immunosuppressive agent” refers to a compound or gene product that has an inhibitory effect on the functions of the immune response or a compound such as a chemical compound, small molecule which possesses immune response inhibitory activity. Examples of immunosuppressive agents include, but are not limited to, steroids (prednisolone, methylprednisolone, etc.), IL-2 antibodies, IL-2 secretion suppressors (rapamycin, etc.), CN suppressors such as cyclosporine or FK506 (also referred to as tacrolimus or Prograf), antimetabolite agents (mycophenolate mofetil, etc.), cyclophosphamide, OKT3 (also referred to as muromonab-CD3), antilymphocytic globulin (antithymocyte immunoglobulin), anti-CD4 antibodies, anti-TNF-a antibodies, azathioprine, mizoribine, sulfasalazine, 6-mercaptopurine (6-MP), methotrexate, cytoxazone, gusperimus hydrochloride, or combinations of these agents.

An “anti-inflammatory drug” includes any substance capable of producing an anti-inflammatory effect, e.g., the prevention or diminution of the inflammation, as by irradiation or by administration of drugs such as anti-TNF drugs.

An “anti-histamine drug” is an agent that inhibits the action of histamine. Such drugs and include agents which block the binding of histamine to histamine receptors and inhibit the acitivity of histidine decarboxylase. They are widely used and well known in the art and include, for example, acrivastine, azelastine, brompheniramine, buclizine, bromodiphenhydramine, carbinoxamine, cetirizine, chlorpromazine, cyclizine, chlorpheniramine, chlorodiphenhydramine, clemastine, cyproheptadine, desloratadine, dexbrompheniramine, dexchlorpheniramine, dimenhydrinate, dimetindene, diphenhydramine, doxylamine, ebastine, embramine, fexofenadine, levocetirizine, loratadine, meclozine, mirtazapine, olopatadine, orphenadrine, phenindamine, pheniramine, phenyltoloxamine, promethazine, pyrilamine, quetiapine, rupatadine, tripelennamine, triprolidine, cimetidine, famotidine, lafutidine, nizatidine, ranitidine and roxatidine.

An “anti-diabetic drug” is an agent used to treat diabetes mellitus by lowering blood glucose levels. Such drugs include insulin; agents that stimulate insulin secretion, including sulfonylurea drugs such as glyburide, glimepiride and glipizide, GLP and GLP agonists/analogues such as exenatude, liraglutide, vildagliptin, sitagliptin, saxagliptin and linagliptin; agents which increase insulin sensitivity, including metformin and thiazolidinediones such as pioglitazone and rosiglitazone; and agents which reduce glucose absorption in the gut, including alpha-glucosidase inhibitors such as acarbose, miglitol and voglibose.

The term “immune disorders” or “immune diseases” or “immune dysregulatory disorder” refers to diseases wherein a reaction of the immune system is responsible for or sustains a malfunction or non-physiological situation in an organism. An immune dysregulatory disorder may be due to inadequate immune response leading to an inflammatory process.

Said dysregulation may be an under-regulation, in the case of immune-deficiency disorders or an over-regulation

An immune over-regulation may include inflammatory disease such as inflammatory bowel disease, atherosclerosis, and autoimmune diseases such as multiple sclerosis, rhumatoid arthritis, and diabetes mellitus, and allergic disorders such as asthma, allergic rhinitis, conjonctivis and atopic dermatitis, alloimmunisation reactions, rejection of viral vectors used in gene therapy/gene vaccination.

For instance, it has been proposed that an imbalance between Th2 and Th1 effectors drives the pathogenesis of asthma.

As used herein, an “autoimmune disease” is a disease or disorder arising from and directed against an individual's own tissues. Examples of autoimmune diseases or disorders include, but are not limited to, arthritis (rheumatoid arthritis, juvenile rheumatoid arthritis, psoriatic arthritis), conditions involving infiltration of T cells and chronic inflammatory responses, autoimmune myocarditis, multiple sclerosis, pemphigus, primary biliary cirrhosis, ANCA vasculitis, autoimmune hepatitis, Lupus, scleroderma and Type 1 diabetes (also referred to as insulin-dependent diabetes mellitus (IDDM)).

As used herein, the term “organ or tissue transplant” refers to any solid organ such as kidneys, heart, lungs, liver, and pancreas, including tissue grafts, and whole or selected populations of blood or bone marrow transplants.

As used herein, the term “Type 1 diabetes” (also referred to as insulin-dependent, juvenile diabetes, or childhood-onset diabetes), refers to an autoimmune disease that results in destruction of insulin-producing beta cells of the pancreas, eventually resulting in a lack of insulin production.

The terms “CD4+ effector cells” “CD4+ CD25− cells” or “effector T cells” or “CD4+ CD25− effector cells” refers to cells belonging to the CD4-positive and CD25-negative subset of T-cells whose function is to provide help to other cells, such as, for example B-cells. These effector cells are conventionally reported as Th cells (for T helper cells), with different subsets such as Th0, Th1, Th2, Th17 cells and T_{FH}. CD4+CD25− T cells may be isolated from a population of T cells, such as isolated peripheral blood lymphocytes, by positive enrichment of CD4+ CD25− T cells. CD4+ CD25− T cells may be isolated using labeled anti-CD4 monoclonal antibody and cell unlabeled anti-CD25 monoclonal antibody.

As used herein, the terms “CD4+CD25+ T cell”, “CD4+CD25+ lymphocyte” “CD4+CD25+ regulatory T cells” or “Treg” refer to any lymphocyte that expresses on its surface the cluster of differentiation markers known as CD4 and CD25 and low expression of CD127. Treg cells are thought to function as a regulator of autoimmunity by suppressing the proliferation and/or cytokine production of CD4+CD25− T cell responder cells at the site of inflammation.

The term “functional CD4+ CD25+ cells” means Treg cells having the capacity to suppress effector T cells function.

“CD4+ CD25+ cell-mediated immunosuppression” means the inhibiting effect of Treg cells on immunity mediated by effector CD4+CD25− T cells. Said inhibiting effect of Treg cells may be a direct effect on CD4+ CD25−, such as inhibition of CD4+CD25− cell proliferation, inhibition of cytokines production, anergy.

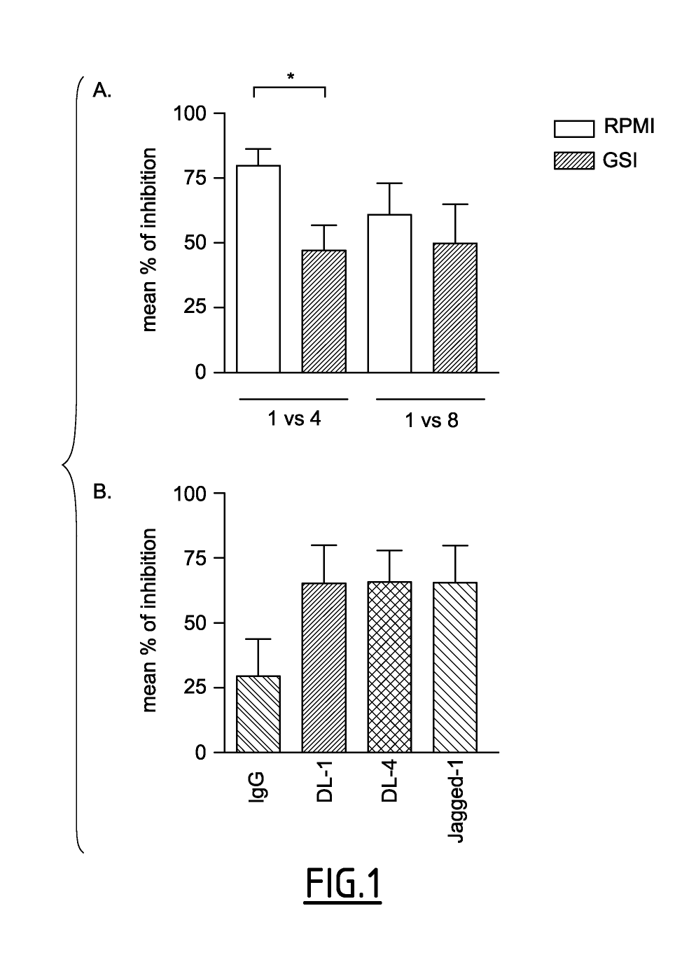

FIG. 1: NOTCH signalling is involved in Treg mediated inhibition of anti-CD3 stimulated CD4⁺CD25⁻ T cell proliferation
A) Various ratio of CD4⁺CD25⁻ T cells and CD4⁺CD25^{high} T cells were cultured for 5 days in coated wells with anti-CD3 (1 μg/ml) in the presence or not of inhibitor of g-secretase (GSI 10 μM). Percentages of inhibition were calculated from the raw data using the equations: 1—(mean cpm of co-culture wells divided by mean cpm of CD4⁺CD25⁻ cells cultured alone)×100. The data represent the mean±SEM and are representative of 5 independent experiments. * indicates P<0.05.
B) CD4⁺CD25⁻T cells were cultured for 18 hours in coated wells with anti-CD3 and NOTCH ligands (5 μg/ml) or IgG control (5 μg/ml). Then, T cells were washed and incubated for 5 days in coated wells with anti-CD3 in the absence or presence of Treg at ratio 1:8. The data represent the mean±SEM of inhibition of T cell proliferation of 5 independent experiments.

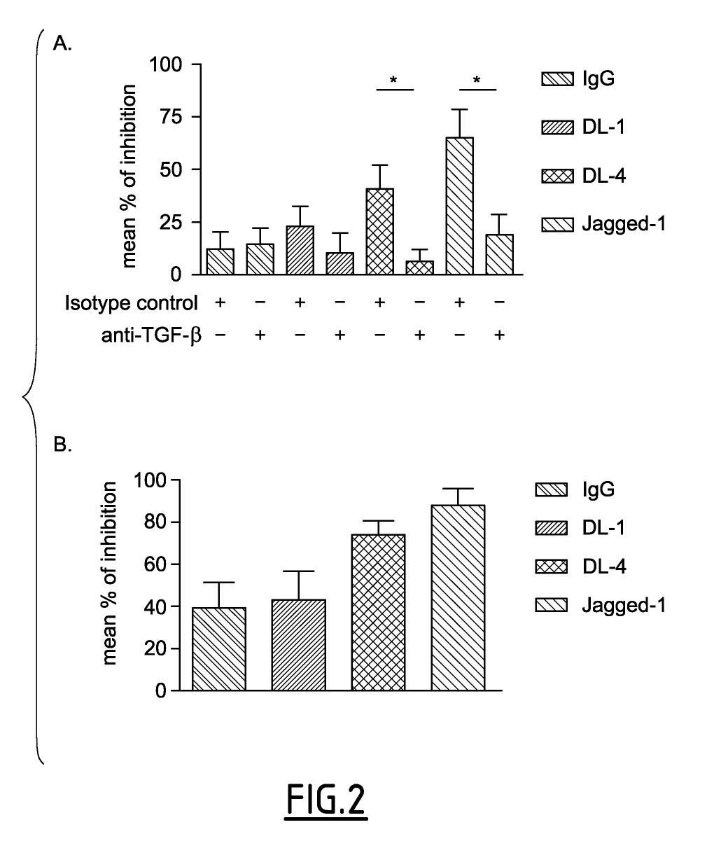

FIG. 2: NOTCH suppresses the proliferation of CD4⁺CD25⁻ T cells through TGF-β
A) CD4⁺CD25⁻T cells were cultured for 5 days in coated wells with anti-CD3 and NOTCH ligands (5 μg/ml) or IgG control (5 μg/ml) in the presence of Treg at ratio 1:16. Anti-TGF-β or IgG control mAbs (2.5 μg/ml) were added at the beginning of the co-culture. The data represent the mean±SEM. * indicates P<0.05.
B) CD4⁺CD25⁻T cells were cultured for 5 days in coated wells with anti-CD3 and NOTCH ligands (5 μg/ml) or IgG control (5 μg/ml). Low dose of TGF-β (0.1 ng/ml) was added. The data represent the mean±SEM.

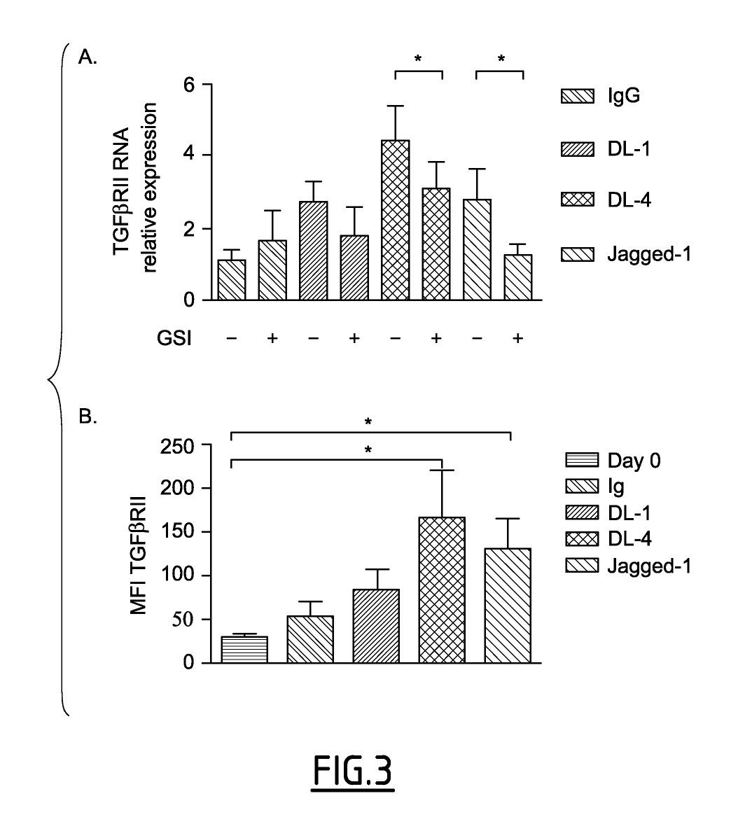

FIG. 3: TGF-βRII expression on effector T cells after NOTCH ligands exposure
A) CD4⁺CD25⁻T cells were purified using Miltenyi microbeads from PBMC and cultured for 3 hours in coated wells with anti-CD3 (200 ng/ml) and NOTCH ligands (5 μg/ml) or IgG control (5 μg/ml). Quantitative real-time PCR was performed to measure the transcripts levels of TGF-βRII. Data shown are mean±SEM and are representative of 4 independent experiments. * indicates P<0.05.
B) Protein levels of TGF-βRII in CD4⁺CD25⁻T cells stimulated to DL-1, DL-4 and Jagged-1 using FACS analysis. Mean of fluorescence are represented±SEM. * indicates P<0.05.

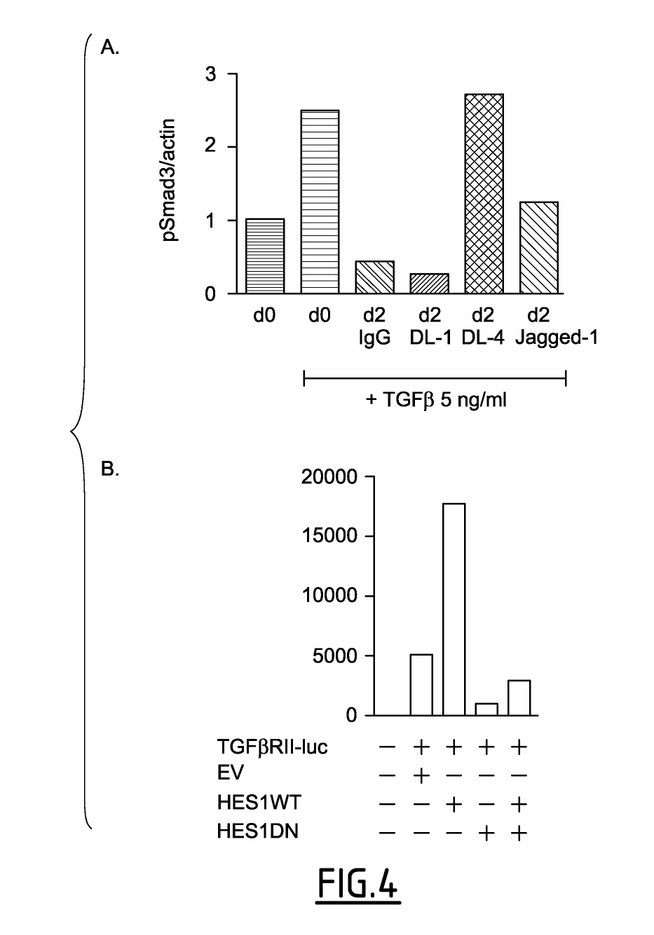

FIG. 4: TGF-βRII function on effector T cells stimulated to NOTCH ligands
A) Western blot analysis was performed on CD4⁺CD25⁻T cells cultured for 48 hours in coated wells with anti-CD3 (200 ng/ml) and NOTCH ligands (5 μg/ml) in presence of TGF-β (5 ng/ml). The whole cell lysate was analysed by immunoblot for pSmad3 and b-actin was used as loading control. The experiment was repeated twice with similar results. Histogram shows quantification data of the western blot results.
B) Equal numbers of PC12 cells were cotransfected with 1 μg of TβRII promoter-luciferase constructs, 1 μg of β-galactosidase and with 50 ng of either pCI-HES-1 or pCI-dnHES-1, or both, or empty vector. Cells were analyzed 48 hours later and data normalized for β-galactosidase activity. Histograms show the luciferase activity in a representative experiment performed in PC12 with TβRII promoter-luciferase construct. The experiment was repeated 3 times with similar results.

### EXAMPLE 1

### Materials and Methods

### Cell Culture

Peripheral blood mononuclear cells were separated by Ficoll-Hypaque centrifugation (Amersham Biosciences) from buffy coats obtained from healthy blood donors (EFS, Créteil). CD4⁺ CD25⁻ T cells and CD4⁺CD25^{high} T cells were isolated using a CD4⁺ T cell enrichment column and a human CD4⁺CD25⁺ Treg isolation kit purchased from Miltenyi Biotec. CD4⁺CD25⁻ cells and CD4⁺CD25⁺ cells were enriched to greater than 95% purity. Cells were plated in media at a concentration of 1.25×10⁵ to 5×10⁵ cells per ml in a 96-well round-bottom plates coated with 1 μg/ml of anti-CD3 (UCHT1) and 5 μg/ml of NOTCH Ligand (DL-1-Fc, DL-4-Fc and Jagged-1-Fc are a generous Gift from Dr. Sakano).

### Suppression Assay

A total of 2.5×10⁴ CD4⁺CD25⁻ T cells were cultured with various ratios of CD4⁺CD25^{high} cells and 1 μg/ml of anti-CD3 for 5 days, in the presence or absence of 10 μm of the gamma-secretase inhibitor Compound E. A total of 0.5 Ci [³H] thymidine (Amersham Pharmacia) was added to the wells during the last 16 hours of culture. The cells were then harvested and assessed for [³H] thymidine incorporation using a liquid scintillation counter. Results were expressed as mean cpm of quadriplicate culture wells.

Percentage of inhibition was calculated as follows: 1−(mean cpm of co-culture wells divided by mean cpm of CD4⁺CD25⁻ cells cultured alone)×100.

In sensitizing assays CD4⁺CD25⁻ and CD4⁺CD25^{high} were pre-incubated during 16 hours on coated NOTCH Ligand. Cells were also collected, washed and incubated alone or with Treg and stimulated as described above. Some experiments were performed in the presence of anti-TGF-β or IgG control (2.5 μg/ml).

### Real-Time PCR

CD4⁺CD25⁻ T cells were cultured for 3 hours in coated wells with anti-CD3 (200 ng/ml) and NOTCH ligands (5 μg/ml) or IgG control (5 μg/ml). Total RNA was extracted with Trizol (Invitrogen) and purified by chloroform extraction. RNA was then reverse transcribed using 1 mM oligodT and the Superscript™ HH Rnase H-reverse transcriptase (Invitrogen Life technologies) according to the manufacturer's instructions. Quantitative PCR was performed in a LightCycler System (Roche diagnostics) using a SYBR Green PCR kit from Roche Diagnostics. The cDNA input for each population was normalized to obtain equivalent signals with Splicing Factor 3A1 (SF3A1) used as housekeeping gene. Primers used were:

| S14: | |
|---|---|
| (SEQ ID NO: 17) | |
| Forward: | 5′ GGCAGACCGAGATGAATCCTCA 3′ |
| (SEQ ID NO: 18) | |
| Reverse: | 5′ CAGGTCCAGGGGTCTTGGTCC 3′ |
| TGF-βRII | |
| (SEQ ID NO: 19) | |
| Forward: | 5′ CTGCAAGATACATGGCTCCA 3′ |
| (SEQ ID NO: 20) | |
| Reverse: | 5′ CTCGATCTCTCAACACGTTGT 3′ |

### Surface Staining

For surface staining, the cells were harvested, washed twice and resuspended in 1% FCS buffer. The cells were stained with mAbs for CD4 (Beckman Coulter), TGF-βRII (R&D) as per manufacturer's instructions for 20 min at 4° C. Cells were then analyzed by flow cytometry using FACScalibur™ and CellQuest™ software (Becton Dickinson, San Jose, Calif., USA).

### Immuno-Blot Analysis

CD4⁺CD25⁻ T cells were stimulated with NOTCH Ligand during 2 days. 1 ng/ml of TGF-β was also added during 30 min to induce Smad3 phosphorylation. Cells were collected, washed and lysed with Tris HCL (20 mM) 0.5% SDS buffer in presence of DNase (benzonase), inhibitors of phosphatase and protease. 10 μg proteins were deposed for Western blot Analysis with following Antibodies: Anti-PhosphoSmad3, Smad3, Actine.

### Transient Transfection and Luciferase Assay.

### Expression Plasmids.

The TGF-βRII promoter-luciferase constructs were described previously, wtHES-1 and dominant negative (DN) HES-1 were provided by R. Kageyama and β-Galactosidase by I. Dusanter-Fourt (INSERM U 567, Paris). The DN HES-1 has three amino acid mutations only in the basic region (DNA-binding domain), so it cannot bind to the DNA or interact with promoters but can dimerize with endogenous wild-type HES-1 to form a non DNA-binding heterodimer complex.

### Transfection Procedure.

Transfections reporter assays were carried out in six-well tissue culture dishes with the indicated plasmids by using the Lipofectamine plus reagent (Life technologies) as indicated by the manufacturer. PC12 cells were seeded the day prior to transfection at a concentration that will give 50% confluency. Transfection was carried out 18 hours in serum free optiMEM, and cells were then incubated another 48 hours in complete medium (RPMI, 10% HS, 5% FCS). For normalization of transfection efficiency, pCMV-β Gal plasmid diluted one-fourth was added as an internal control.

### EXAMPLE 2

### CD4+CD25− T Cells Stimulated with NOTCH Ligands are More Sensitive to Treg-Mediated Suppression

In order to investigate the involvement of NOTCH signalling in Treg-mediated suppression, a co-culture experiments of purified CD4⁺CD25^{high} and autologous CD4⁺CD25⁻ T cells stimulated with anti-CD3 mAbs was performed in the presence or absence of a g-secretase inhibitor (GSI) of NOTCH signalling.

As shown by FIG. 1A, CD4⁺CD25⁻ T cell proliferation was inhibited (mean+/−SD) by 79.2%+/−12.2 and 60.2%+/−24.8 when cultured with Treg at a ratio (Treg/Effector) 1:4 and 1:8, respectively (mean of 4 experiments). While GSI did not affect the proliferation of CD4⁺CD25⁻ T cells cultured alone (data not shown), addition of this compound at the beginning of the coculture relieved the Treg-mediated suppression which became 46.9%+/−20.5 (P<0.05 for comparison with coculture performed with GSI vehicle) and 49.3%+/−21 at the corresponding ratios.

### EXAMPLE 3

### NOTCH Signalling Sensitizes Effector T Cells to Treg-Mediated Suppression

In order to investigate whether NOTCH signalling increases the sensitivity of effector T cells to Treg suppression, an assay where effector T (CD4⁺CD25⁻) cells were preincubated with recombinant NOTCH ligands, DL-1, DL-4, Jagged-1 or IgG control overnight was performed. Then, T cells were washed and incubated for 5 days in the presence of anti-CD3 with or without Treg. As shown in FIG. 1B, CD4⁺CD25⁻ T cells pre-exposed to DL-1, DL-4 or Jagged-1 exhibit a higher sensitivity to the suppressive effects of Treg. Percentages of inhibition of cell proliferation were 64.8, 64.2, 65% (mean of 4 experiments at a ratio 1:8) in cocultures performed with Treg and CD4⁺CD25⁻ T cells pre-exposed to DL-1, DL-4 and Jagged-1, respectively as compared to culture conditions with effector T cells pre-incubated with IgG control (29.2%) (P<0.05 for all comparisons to IgG control). Exposure of Treg to DL-1, DL-4 or Jagged-1 did not modify the Treg capacity to suppress CD4⁺CD25⁻ proliferation in the presence of anti-CD3 mAbs (data not shown). These results show that NOTCH signalling acts on effector T cells and that NOTCH ligands potentiate Treg-mediated suppression.

### EXAMPLE 4

### NOTCH Suppresses the Proliferation of CD4+ CD25− T Cells Through TGF-β Signalling

The implication of NOTCH1 on effector T cell responses in the presence of Treg through a TGF-β signalling mechanism has not yet been elucidated. To test this, a coculture experiments of effector T cells and Treg at a low ratio (ratio 1:16) in the presence of immobilized NOTCH ligands DL1, DL4 and Jagged-1 or Ig controls at 5 mg/ml was performed. Anti-TGF-β (2.5 mg/ml) or isotype control mAbs were added at the beginning of the culture. Coculture experiments performed in the presence of either DL-4 or Jagged-1 led to a mean inhibition of T cell proliferation of 47% and 65%, respectively as compared to 12% when cells were pre-treated with IgG controls. As shown in FIG. 2A, these percentages became 6.3% and 19.3% in the presence of anti-TGF-β mAbs (P<0.05) while no changes were noted in the presence of isotype control. Finally, anti-TGF-β did not affect coculture performed with Treg and effector T cells pre-treated with DL-1 ligands. Next, the impact of NOTCH on the sensitization of effector CD4⁺CD25⁻ T cells to TGF-β was investigated. To this end, purified CD4⁺CD25⁻ T cells were stimulated with anti-CD3 antibodies and either NOTCH ligands or IgG control in the presence of low concentration of TGF-β (0.1 ng/ml). As shown in FIG. 2B, TGF-β increased the percentage of inhibition in cultures performed with DL-4 (74.1%) and Jagged-1 (87.8%) compared to DL-1 (42.9%) and IgG control (38.9%) (P<0.05). These results suggest that NOTCH signalling increases the sensitivity of effector T cells to TGF-β mediated suppression.

### EXAMPLE 5

### Regulation of TGF-βRII Expression by NOTCH Ligands

The above experiments show that NOTCH signalling increased responses of effector T cells to low amounts of TGF-β. Active TGF-β mediates its biological functions by binding to TGF-β type I and type II receptors (TGF-βRII). Therefore, the effects of NOTCH activation on TGF-βRII expression of effector T cells were first investigated. As shown in FIG. 3A, CD4⁺CD25⁻ effector T cells stimulated with coated anti-CD3 and NOTCH ligands (DL-4 or Jagged-1) for 3 hours, exhibited a marked increase in TGF-βRII RNA expression as compared to pre-exposure to IgG control (4.4 and 2.8 fold increase for DL-4 and Jagged-1, respectively). This effect is abrogated in the presence of GSI (P<0.05 for comparison with culture performed without GSI vehicle). According to the results presented above, pre-exposure of effector T cells to DL-1 alone, or in the presence of GSI, did not significantly modify TGF-βRII RNA expression.

TGF-βRII protein expression was assessed by flow cytometry. In these experiments, effector CD4⁺CD25⁻ T cells were isolated and incubated for 48 hours in the presence of NOTCH ligands or IgG control. Flow analysis showed that DL-4 and Jagged-1 increased expression of TGF-βRII (MFI: 166 and 130 respectively) as compared to IgG (MFI: 53) or DL-1 (MFI: 85) (FIG. 3B). Together these results suggest that engagement of DL-4 and Jagged-1 increase TGF-βRII expression on CD4⁺CD25⁻ T cells which may facilitate TGF-β mediated effector function of Tregs.

### EXAMPLE 6

### DL-4 and Jagged-1 Sustain TGF-βRII Function on CD4+CD25−T Cells Through HES Interaction with the TGF-βRII Promoter

Given the above observations showing an upregulation of TGF-βRII following NOTCH activation, the inventors hypothesized that Jagged-1 and DL-4 led to an activation of TGF-β signalling in effector T cells. CD4⁺CD25⁻ T cells were isolated and cultured for 48 hours in wells coated with NOTCH ligands or IgG control in the presence of TGF-β (5 ng/ml). An immunoblot was performed to assess the expression of pSmad3, a major TGF-β signalling intermediate (Li, M. O. et al., 2006. Annu Rev Immunol 24:99). The immunoblot was then quantified and the resulted histogram is shown in FIG. 4A. According to these experiments, anti-pSmad3 revealed a strong pSmad3 signal in DL-4 and Jagged-1 conditions, while only a weak pSmad3 signal was detected in IgG and DL-1 stimulated cells.

Transactivation experiments were performed to explore the mechanism by which NOTCH may regulate TGF-βRII expression. Since these experiments cannot be performed easily on primary CD4⁺ T cells, rat PC12 cells were used. Rat PC12 cell lines are widely used to explore the role of HES-1 as a modulator of cell differentiation and proliferation (Castella, P. et al., 2000. Mol Cell Biol 20:6170). The transactivation of the TGF-βRII promoter by wtHes-1 (pCI-HES-1) or a DN mutant (dnHES-1) was analyzed in three separate experiments. WtHES-1 was found to induce a strong transactivation of the TGF-βRII luciferase reporter promoter containing 5′ sequences compared with transfection with the empty vector (EV) (FIG. 4B). No increase in luciferase activity was seen when wtHES-1 and dnHES-1 were cotransfected. Interestingly, when transfected alone, the dnHES-1 plasmid decreased luciferase activity below that measured with the empty vector, since it neutralized endogeneous HES-1 protein activity on the TGF-βRII reporter promoter. These results demonstrated that HES-1 activates the TGF-βRII promoter through its DNA-binding activity.

1. A method for sensitizing CD4+ CD25− cells to CD4+ CD25+ cell-mediated immunosuppression, wherein said method comprises administering to an individual a NOTCH agonist.

2. The method according to claim 1, wherein said method is a method for enhancing the expression of the TGF-β receptor in CD4+ CD25− cells.

3. The method according to claim 2, wherein said TGF-β receptor is TGF-β type II receptor.

4. The method according to claim 1, wherein said method is a method of prevention and/or treatment of an immune dysregulatory disorder.

5. The method according to claim 4, wherein said immune dysregulatory disorder is an autoimmune disease, inflammatory disorder, cardio-vascular disease or diabetes.

6. The method according to claim 1, wherein said NOTCH agonist is selected from the group consisting of Delta-like 4, Jagged-1 and a biologically active fragment thereof.

7. The method according to claim 1, wherein the NOTCH agonist is administered in combination with a product selected from the group consisting of anti-diabetes and immunomodulatory drugs, either simultaneously, separately or sequentially.

8. The method according to claim 7, wherein said immunomodulatory drug is selected from the group consisting of anti-histamine and anti-inflammatory drugs.

9. An in vitro or ex vivo method for sensitizing CD4+ CD25− cells to CD4+ CD25+ cell-mediated immunosuppression comprising the steps of:
a) providing a biological sample comprising CD4+ CD25− cells; and
b) contacting the biological sample with a NOTCH agonist.

10. An in vitro or ex vivo method of selecting a patient suffering from an immune dysregulatory disorder to be treated by a NOTCH agonist which method comprises the steps of:
a) providing a biological sample comprising CD4+ CD25− cells from said patient;
b) determining whether:
said CD4+ CD25− cells are resistant to CD4+ CD25+ cell-mediated immunosuppression; and/or
TGF-β receptor is expressed on said CD4+ CD25− cells; and
c) selecting said patient for treatment by a NOTCH agonist if said CD4+ CD25− cells are CD4+ CD25+ cell resistant, and/or if TGF-β receptor is expressed at a significantly lower level in said biological sample than in a control sample.

11. The method according to claim 4, wherein said method is a method of prevention and/or treatment of an immune dysregulatory disorder of an individual having CD4+ CD25− cells which are resistant to CD4+ CD25+ cell-mediated immunosuppression; and/or which under express TGF-β receptor.

12-13. (canceled)
